(19) United States
(12) Patent Application Publication
(10) Pub. No.: US 2024/0327332 A1
(43) Pub. Date: Oct. 3, 2024

(54) Process for Producing NAA and (S)-NAA

(71) Applicant: Syngenta Crop Protection AG, Basel (CH)

(72) Inventors: George Robert Hodges, Bracknell (GB); Martin Charles Bowden, Bracknell (GB)

(73) Assignee: Syngenta Crop Protection AG, Basel (CH)

(21) Appl. No.: 18/550,336

(22) PCT Filed: Mar. 23, 2022
(86) PCT No.: PCT/EP2022/057617

§ 371 Date: Sep. 13, 2023

(30) Foreign Application Priority Data
Mar. 29, 2021 (GB) 2104413.6

(51) Int. Cl. C07C 213/08

(52) U.S. Cl. CPC C07C 213/08

(57) ABSTRACT

A method of producing a compound formula (I)

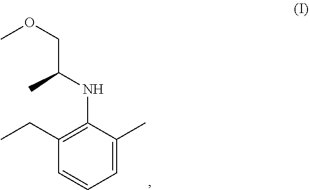

the method comprising: reacting a compound of formula (II)

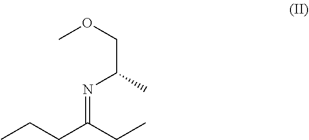

with a compound of formula (III)

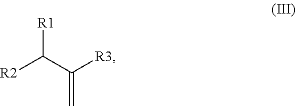

wherein,
R3 is hydrogen or halogen;
R1 and R2 are each independently C₁-C₆alkoxy, or R1 and R2 together are ═O or —OCH₂CH₂O— or —OCH₂CH₂CH₂O—; and
optionally whereby the reaction is carried out in the presence of an oxidizer.

S-Metolachlor (S-MOC) and metolachlor are part of the chloroacetanilide family of herbicides, used to control grasses and broad-leafed weeds in maize. The (S) enantiomer of metolachlor is approximately twenty times more active than the (R) enantiomer.

S-Metolachlor and metolachlor are known to be produced by reacting (S)-NAA or NAA and with chloroacetyl chloride as shown below:

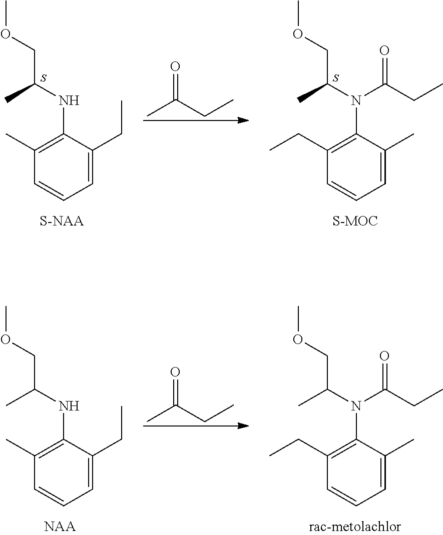

It is known to be manufactured by an iridium-based asymmetric hydrogenation process, which uses a significant fraction of the world's iridium production. Iridium metal is thought to be the rarest stable element in the earth's crust. Iridium is therefore extremely expensive and susceptible to shortages and price fluctuations. Therefore, there is a need for manufacturing processes to produce the (S) enantiomer of metolachlor without the use of Iridium.

To this end, a novel Iridium-free route starting with commercially available (S)-1-methoxy-2-propylamine is presented herein. The novel synthetic pathway provides for a convergent synthesis with potentially only water and HCl as waste products. Furthermore, an advantage of the is process is that it uses conventual equipment known and readily available to those skilled it the art.

There is therefore provided a method of producing a compound formula (I)

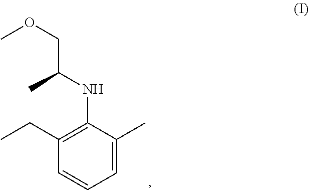

the method comprising: reacting a compound of formula (II)

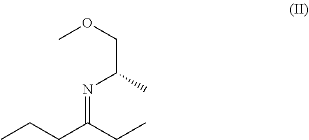

with a compound of formula (III)

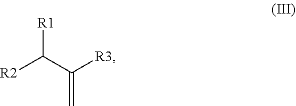

wherein,
R3 is hydrogen or halogen;
R1 and R2 are each independently C₁-C₆alkoxy, or R1 and R2 together are ═O or —OCH₂CH₂O— or —OCH₂CH₂CH₂O—; and
optionally whereby the reaction is carried out in the presence of an oxidizer.

Preferably, R3 is Cl or Br; and/or R1 and R2 are each independently dimethoxy, diethoxy, ethylene glycol, or R1 and R2 together are ═O.

Advantageously the compound of formula (III) is

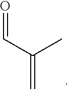

Alternatively, the compound of formula (III) is acrolein or acrolein dimethyl acetal.

In certain embodiments, the present technology may be carried out in the presence of oxidizer, commonly referred to as an oxidant/oxidation catalyst. Typical examples include (by way of example, and not by limitation): oxidising metal salts (preferably salts such as copper (II) acetate), halogenation (with reagents like Cl₂ or Br₂), dehydrogenation by heating with a precious metal catalyst, such as Pd or Pt. Persons skilled in the art will further understand additional ways to convert dihydro-anilines to the corresponding aniline.

There is also provided a method comprising: reacting a compound formula (I)

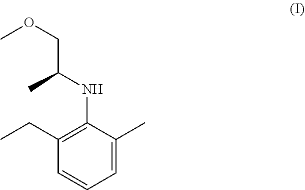

with chloroacetyl chloride, where the compound of formula (I) was produced by the method described herein.

There is provided a method of producing a compound formula (IV)

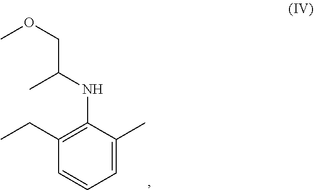

the method comprising: reacting a compound of formula (V)

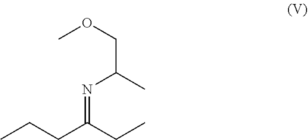

with a compound of formula (VI)

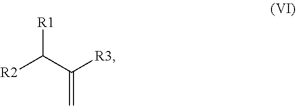

wherein,
R3 is hydrogen or halogen;
R1 and R2 are each independently C₁-C₆alkoxy, or R1 and R2 together are ═O or —OCH₂CH₂O— or —OCH₂CH₂CH₂O—; and
optionally whereby the reaction is carried out in the presence of an oxidizer.

There is also provided a method comprising: reacting a compound of formula (IV)

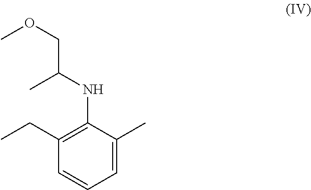

with chloroacetyl chloride, where the compound of formula (IV) was produced by the method described above.

While the present technology provides for a route comprises three steps if the intermediate dihydro-aniline is isolated, it is also envisioned that other dihydro-aniline systems indicates that such oxidation steps can sometimes be conducted in situ. This would reduce the synthesis to two steps.

The invention is demonstrated by the following non-limiting Examples.

### EXAMPLES

The novel approach of the present invention is based on de-novo ring synthesis of the Metolachlor and (S)-Metolachlor intermediate NAA and (S)-NAA, respectively. One embodiment of the de-novo ring synthesis (S)-NAA shown below in Scheme 1(a).

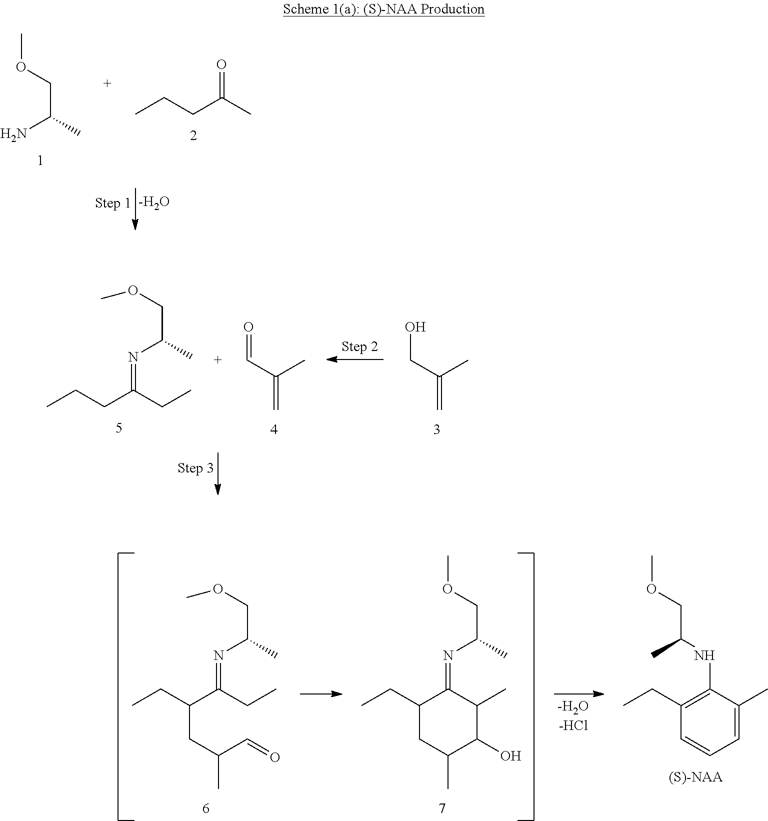

In a further embodiment, the de-novo ring synthesis of (S)-NAA is shown below in Scheme 1(b)

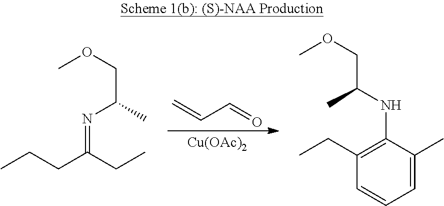

A 100 mL round bottomed flask was equipped with magnetic follower and condenser. To this was added (S)—N-(2-Methoxy-1-methyl-ethyl)hexan-3-imine) (227.6 mg, 1.33 mmol, 1 eq) as a solution in Toluene (6 mL), followed by acrolein (74.5 mg, 88.9 uL, 1.33 mmol, 1 eq). Cu(OAc)₂ (265.5 mg, 1.33 mmol, 1 eq) was added in one portion, followed by an additional 6 mL of toluene. The mixture was warmed to ˜25° C. internal temperature before the dropwise introduction of trifluoroacetic acid (151.7 mg, 102.9 uL, 1.33 mmol, 1 eq). The reaction was then heated to 110° C. (internal temperature) (˜140° C. heating block temperature) maintained at this temperature for 3 hours.

The reaction mixture was allowed to cool to ambient, then concentrated in vacuo. The product (S)-NAA [S-2-Ethyl-N-(2-methoxy-1-methyl-ethyl)-6-methyl-aniline] was formed in 60% conversion, and chiral analysis showed desired S-NAA product to have been formed with >99% e.r.

Imine hydrolysis is problematic, so water removal during the process is desirable. Chemical dehydration using Ac₂O is unsuccessful and use of Na₂SO₄ and MgSO₄ gives only trace levels of product. However, much better results are obtained with molecular sieves as a water scavenger and the reaction of acrolein with the imine in the presence of molecular sieves does not require acid such as TFA. Intact molecular sieve beads perform better than crushed beads/powder.

In a further embodiment, the de-novo ring synthesis of (S)-NAA is shown below in Scheme 1(c):

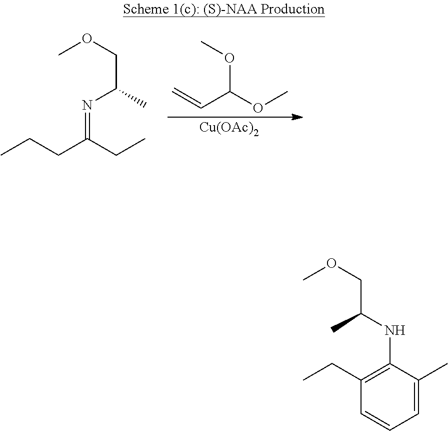

A 25 mL round bottomed flask was equipped with magnetic follower and condenser. To this was added (S)—N-(2-Methoxy-1-methyl-ethyl)hexan-3-imine) (451 mg, 2.63 mmol, 1 eq) as a solution in toluene (10 mL). Acrolein dimethyl acetal (268.8 mg, 311.8 uL, 2.63 mmol, 1 eq) was charged to the reactor as a solution in toluene (6 ml) and the mixture stirred. Cu(OAc)₂ (525 mg, 2.63 mmol, 1 eq) was charged, followed by the dropwise addition of trifluoroacetic acid (200 uL, 2.63 mmol, 1 eq). The reaction was then heated to 110° C. (internal temperature) (˜140° C. heating block temperature) and maintained at this for 60 minutes.

The reaction mixture was allowed to cool to ambient, then concentrated in vacuo. Purification by Combiflash chromatography afforded the desired product S-NAA [S-2-Ethyl-N-(2-methoxy-1-methyl-ethyl)-6-methyl-aniline] as a brown oil with >99% e.r. in 18% isolated yield.

### METHOD/EXPERIMENTAL

### Materials

Molecular sieves (beads, 3 Å, Sigma Aldrich)
Dimethylformamide (anhydrous, 99.8%, Sigma Aldrich)
Hexan-3-one (98%, Sigma Aldrich)
Dichloromethane (99%, Fisher)
2-Amino-1-methoxypropane (95%, Sigma Aldrich)
Toluene (99%, Fisher)
Acetonitrile (99%, Fisher)
Trifluoroacetic acid (99%, Sigma Aldrich)
Hydrochloric acid (2M, Fisher)
Oxalyl chloride (98%, Sigma Aldrich)
2-chloropropen-1-ol (CPol) (90%, Sigma Aldrich)
Dimethyl Sulfoxide (anhydrous, 99.9%, Sigma Aldrich)
Triethylamine (99.5%, Fisher)
1,3,5-Trimethoxybenzene (99%, Fisher)
Acetic acid (99%, Sigma Aldrich)
Titanium (IV) isopropoxide (97%, Sigma Aldrich)
Potassium carbonate (99.9%, Sigma Aldrich)
Ethyl acetate (99%, Fisher)
DBU (98%, Sigma Aldrich)
Proline (95%, Sigma Aldrich)
Sodium acetate (99.5%, Sigma Aldrich)
Methanesulfonic acid (98%, Fisher)
Hexafluoroisopropanol (98%, Fisher)
Tetrabutylammonium acetate (Anhyrous, 90%, Fisher)
NMP (Anhydrous, 99.8%, Sigma Aldrich)
Propylene carbonate (Anhydrous, 99%, Sigma Aldrich)
Benzoic acid (98%, Fisher)
Monosodium phosphate (98%, Sigma Aldrich)

### Experiment 1

### Step 1: N-(2-Methoxy-1-methyl-ethyl)hexan-3-imine

An oven dried 3-necked round-bottomed flask equipped with thermometer was purged with inert gas. A Dean stark trap equipped with condenser (filled with molecular sieves and reaction solvent filled to the arm of the trap) was attached. Solvent (10-150 mL, toluene) was added. The ketone (2-75 mmol) and amine (4-150 mmol, 2.0 eq) were then charged through seal. Reaction stirred at room temperature for 10 min before addition of acid 0.01-1.0 eq). The reaction was stirred at room temperature for 10 mins before heating to 105° C. internal temperature. The reaction was observed to be at steady state for 30 min before stirring at temperature for the reaction time. Reaction may be sampled into GC vials. The vessel was cooled to ambient, either A: the solution neutralised with potassium carbonate if necessary, filtered and the product extracted in toluene if necessary (3×100 mL) or B: addition of water followed by phase separation via liquid liquid extraction or phase separator membrane. The solvent was then dried followed by azeotroping with acetonitrile to remove residual toluene.

### Step 2: 2-Chloroacrolein

An oven dried round bottom flask equipped with nitrogen inlet and bleach scrubber outlet was cooled in an IPA cardice bath to −78° C. Oxalyl chloride (2.2-66 mmol, 1.1 eq) in DCM (2-20 mL) was added, followed by addition of a solution of DMSO (2.2-66 mmol, 1.1 eq) in DCM (2-20 mL) dropwise over 5 min. The solution was stirred for 15 min, followed by warming to −60° C., stirred for 15 mins and cooled to −78° C. 2-chloroprop-2-en-ol (CPol, 2-60 mmol) in DCM (2-20 mL) was added dropwise, followed by allowing the mixture to warm to −50° C. Trimethylamine (5.0 eq) was added, and the reaction held at −50° C. with stirring for 30 mins, followed by allowing the reaction to warm to ambient over 2 hr. The mixture was acidified (1-3 M HCl, 10-130 mL) and the product extracted in DCM (3×30 mL. The solvent was removed at reduced pressure (200 mbar, 30° C.). The product was stored immediately at −20° C. with 10 mol % hydroquinone stabiliser. Reaction glassware cleaned with bleach to avoid dimethyl sulphide vapour escaping.

### Step 3:2-Ethyl-N-(2-methoxy-1-methyl-ethyl)-6-methyl-aniline [NAA]

### Base Catalysed

An oven dried round bottom flask equipped with nitrogen inlet was heated or cooled as appropriate in the range 0-80° C. with stirring. Imine (1-20 mmol), in solvent (5-20 mL) was added, followed by 2-chloroacrolein (1-20 mmol, 1-2.0 eq.) in solvent (5-20 mL). Base (1-25 mol %) was added and the mixture stirred for 2-24 hr, followed by warming or cooling to room temperature. The mixture was acidified (1 M HCl, 10-50 mL) and the product extracted in DCM (3×30 mL). The solvent was removed at reduced pressure, and further purification completed as necessary.

### Acid Catalysed

An oven dried round bottom flask equipped with nitrogen inlet was heated as appropriate in the range 25-115° C. on a drysyn heating block with stirring. Foil was added for all temperatures above 100° C. and condenser used if within 25° C. of lowest boiling component. Imine (1-20 mmol) in solvent (5-20 mL) was added, followed by 2-chloroacrolein (1-20 mmol, 1-2.0 eq) in solvent (5-20 mL). Acid catalyst (0-150 mol %) was added and the mixture stirred for 2-24 hr, followed by cooling to room temperature. The reaction was neutralised as necessary with dilute base (10-30 mL), and the mixture was extracted and concentrated under vacuum, which was followed by purification by Combiflash if necessary. Dean stark apparatus or molecular sieves (10% w/v) may be added to remove water from the reaction flask

### Experiment 2

### Step 1: N-(2-Methoxy-1-methyl-ethyl)hexan-3-imine

An oven dried 3 necked RB flask equipped with thermometer and magnetic follower was purged with inert gas. A Dean stark trap equipped with condenser (filled with molecular sieves and reaction solvent filled to the arm of the trap) was attached. Solvent (150 mL, aim for 60% of reactor volume) was added. The ketone (75 mmol, approx 5% v/v) and amine (150 mmol, 2.0 eq) were then charged through seal via syringe. Reaction stirred at room temperature for 10 min before addition of TFA (0.01 eq). The reaction was stirred at room temperature for 10 mins before heating to 105° C. internal temperature. The reaction was observed to be at steady state for 30 min before stirring at temperature for the reaction time, ensure condensation of water and solvent occurs before leaving to stir. The vessel was cooled to ambient, the solution neutralised with potassium carbonate (0.02 eq), magnesium sulfate was added, and then filtered. The solvent was removed (35 mbar, 30° C.) before azeotroping with acetonitrile (3:1 ratio acetonitrile:toluene, 2 repeats) to remove residual toluene. The product was decanted off from the viscous orange oil (assumed to be oligomer and salts) Product was stored over molecular sieves. Yield 45%, (strength 85%).

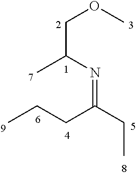

¹H NMR, (400 MHZ, d8-toluene) δ: 3.97 (tq, CH, 1H, H-1), 3.33 (d, CH₂, 1H, H-2a), 3.31 (d, CH₂, 1H, H-2b), 3.14 (obs d (isomers), OCH₃, 3H, H-3a&b), 2.11 (t, CH₂, 2H, H-4), 2.05-1.98 (m, CH₂, 2H, H-5), 1.62 (sext, CH₂, 1H, H-6a), 1.37-1.29 (m, CH₂, 1H, H-6b), 1.14-1.10 (m, CH₃, 3H, H-7), 0.93-0.86 (m, CH₃, 3H, H-8), 0.85-0.74 (m, CH₃, 3H, H-9). ¹³C NMR, (400 MHZ, d8-toluene) δ: 171.0, 170.8 (isomer, imine), 78.9 (obs d, C-2), 58.7 (obs d, C-3), 54.7 (obs d, C-1), 41.3 (C-4), 33.6 (C-8), 32.8 (C-6), 24.5 (C-8), 19.3 (C-7), 14.4 (C-9).

### Step 2:2-Chloroacrolein

A round bottomed flask equipped with low temperature thermometer, magnetic follower and nitrogen inlet was flushed with N₂ using needle attachment. A Dreschel bottle of bleach was filled and flow checked through both bubblers. Cardice bath was cooled to −78 C with IPA. Oxalyl chloride (2.2 mmol, 1.1 eq) was added to DCM (5 mL) in a round bottom flask, mixed and then syringed into the reactor. DMSO (2.2 mmol, 1.1 eq) was added to DCM (5 mL), mixed and added dropwise. ensuring internal temperature does not exceed −60° C. Dependent on scale this can take 3-20 mins. The reaction was held at −60° C. for 15 min before cooling to −78° C. Chloropropenol (2 mmol) was added to DCM (5 mL), mixed and added dropwise ensuring temperature does not exceed −60° C. The mixture was allowed to warm to −50° C. Additional dry ice was added to the cooling bath to reduce external temperature to ca −65° C. NEt₃ (10 mmol, 5.0 eq) was added dropwise, ensuring internal temperature did not exceed −45° C. Reaction was observed to be in range −60° C. to −45° C. for 20 minutes followed by warming to ca. −10° C. over 90 min. The reaction was Acidified with HCl (6 eq, 1-3 M), extracted into DCM, washed with water, re-extracted into DCM, dried and filtered. Hydroquinone (5% w/v) was added and the solvent removed via rotary evaporator (200 mbar, 30° C.) for as little time as possible to reduce polymerisation. Product was immediately stored at −20° C. Yield 35% (strength 73%) ¹H NMR (400 MHZ, CDCl₃) δ: 9.46 (s, 1H), 6.59 (d, 1H), 6.42 (d, 1H). ¹³C NMR (400 MHZ, CDCl₃) δ: 185.3, 140.8, 131.9.

### Step 3:2-Ethyl-N-(2-methoxy-1-methyl-ethyl)-6-methyl-aniline [NAA]

An oven dried round bottom flask equipped with nitrogen inlet and magnetic follower was charged with 1,3,5-trimethoxybenzene (10 mol %) internal standard and activated molecular sieves (5% w/v). Imine (15 mmol) was added to toluene (dried over molecular sieves, 20 mL) and mixed before syringing into the reactor. Chloroacrolein (15 mmol, 1.0 eq) was added to toluene (dried over molecular sieves, 20 mL) and syringed into the reactor. Acetic acid (1.0 eq) was added dropwise over ca. 30 seconds if possible, and the reaction heated to 95° C. (external temperature) on a drysyn heating block with stirring for 3 hr. The reaction was cooled to room temperature and concentrated under vacuum (35 mbar, 30° C.) followed by analysis by GC and ¹H NMR. Yield 10% (Strength 9%)

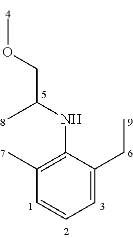

¹H NMR (400 MHz, CDCl₃) δ: 7.01 (d, H-1, 1H), 6.98 (d, H-2, 1-H), 6.86 (t, 1-H, H-3), 3.36 (s, H-4, 3H), 3.32 (m, 3H, H-5), 2.64 (q, 2H, H-6), 2.28 (s, 3H, H-7), 1.22 (t, 2H, H-9), 1.17 (d, 3H, H-8).

### Experiment 3

### Step 1: (S)—N-(2-Methoxy-1-methyl-ethyl)hexan-3-imine

An oven dried 3-necked round bottomed flask equipped with thermometer, magnetic follower and Dean-Stark trap with condenser (filled to the arm of the trap with molecular sieves and reaction solvent). The system was purged with nitrogen, then toluene (150 mL, ca. 60% of reactor volume) added. The ketone (56.09 mmol, approx 5% v/v) and amine (112.18 mmol, 2.0 eq) were charged and the reaction stirred at ambient temperature for 10 minutes. TFA (0.01 eq) was added, and the mixture stirred for a further 10 mins at ambient before heating to 110° C. After reaction completion, the vessel was cooled to ambient, and the solvent then removed in vacuo (35 mbar, 30° C.). Acetonitrile was added to the crude product then removed in vacuo to remove residual toluene. Yield of chiral imine 82%, 93.4% strength, >99% e.r.

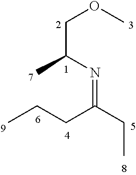

¹H NMR, (400 MHZ, d₈-toluene) δ: 3.97 (tq, 1H, H-1), 3.33 (d, 1H, H-2a), 3.31 (d, 1H, H-2b), 3.14 (obs d (isomers), 3H, H-3a&b), 2.11 (t, 2H, H-4), 2.05-1.98 (m, 2H, H-5), 1.62 (sext, 1H, H-6a), 1.37-1.29 (m, 1H, H-6b), 1.14-1.10 (m, 3H, H-7), 0.93-0.86 (m, 3H, H-8), 0.85-0.74 (m, 3H, H-9).

¹³C NMR, (400 MHZ, d8-toluene) δ: 171.0, 170.8 (isomer, imine), 78.9 (obs d, C-2), 58.7 (obs d, C-3), 54.7 (obs d, C-1), 41.3 (C-4), 33.6 (C-5), 32.8 (C-6), 24.5 (C-8), 19.3 (C-7), 14.4 (C-9),

### Step 3: (S)-2-Ethyl-N-(2-methoxy-1-methyl-ethyl)-6-methyl-aniline [S-NAA]

An oven-dried round bottom flask was equipped with nitrogen inlet and magnetic follower. 1,3,5-trimethoxybenzene (10 mol %) charged as internal standard followed by activated molecular sieves (5% w/v). Imine (15 mmol) was added to anhydrous toluene (dried over molecular sieves, 20 ml), mixed and the solution then syringed into the reactor. Chloroacrolein (15 mmol, 1.0 eq) was dissolved in anhydrous toluene and the solution syringed into the reactor. Acetic acid (1.0 eq) was added dropwise over ca. 30 seconds, the reaction then heated to 95° C. and held with stirring for 3 hr. The reaction was cooled to room temperature and concentrated under vacuum (35 mbar, 30° C.). Analysis by GC and ¹H NMR showed the desired product S-NAA had been formed with >99% e.r, in 12% yield.

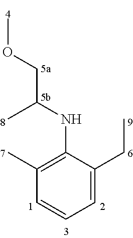

¹H NMR (400 MHZ, CDCl₃) δ: 7.01 (d, H-1, 1H), 6.98 (d, H-2, 1H), 6.86 (t, H-3, 1H), 3.36 (s, H-4, 3H), 3.32 (m, H-5a & b, 3H), 2.64 (q, H-6, 2H), 2.28 (s, H-7, 3H), 1.22 (t, H-9, 2H), 1.17 (d, H-8, 3H).

### Racemic NAA Synthesis

The present technology also provides for a route to produce racemic NAA as provided below in Scheme 2.

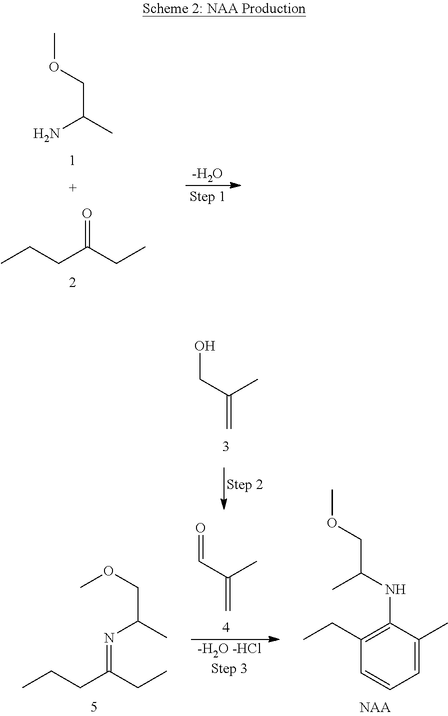

### Step 1: Imine Synthesis

This step involves condensation of 1-methoxy-2-propylamine (1) and 3-hexanone (2) (Scheme 3). Imine formations are typically Lewis or Bronsted acid catalysed and use water removal to push the equilibrium to the desired imine product.

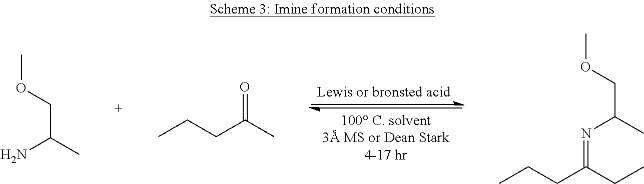

### Titanium Isopropoxide and Acetic Acid:

Titanium isopropoxide was an effective catalyst for this reaction, generating high conversions (by GC) to the desired product (confirmed by ¹H, ¹³C NMR and GC-MS data). The rate of reaction and conversion achieved increased with the amount of catalyst added (Table 1).

TABLE 1
Initial acid-catalysed attempts to form Imine

| Catalyst | | Drying agent | Conversion at end of reaction |
|---|---|---|---|
| (eq) | Solvent | (in reactor) | (%, GC area) |
| AcOH (1.0) | EtOAc | 10 wt % 3 Å MS | 4 |
| TiOiPr₄ (0.1) | EtOAc | 10 wt % 3 Å MS | 33 |
| TiOiPr₄ (0.2) | Toluene | 10 wt % 3 Å MS | 67 |
| TiOiPr₄ (0.5) | Toluene | 10 wt % 3 Å MS | 78 |
| TiOiPr₄ (1.0) | Toluene | 10 wt % 3 Å MS | 96 |

Column chromatography or filtration through an alumina plug resulted in imine hydrolysis with no product recovery. Aqueous workup at room temperature was and improved methodology, but still caused imine hydrolysis issues. The work-up involved addition of water to convert titanium isoproproxide catalyst to titanium dioxide, which was removed by filtration, then solvent removal in vacuo.

This process gave a 22% isolated yield of desired imine product, with a 63% strength by GC. Product strength excluding toluene was 83%.

Isolated Imine showed reasonable stability (stored over activated molecular sieves at room temperature), with ca. 3% hydrolysis per week being observed reducing to the equilibrium point of approximately 65%. Older samples may be able to be ‘reactivated’ by further distillation of starting materials after hydrolysis.

### Un-Catalysed Reaction:

Despite the high conversions achieved, the workup issues with Ti(OⁱPr)₄ prompted an investigation of the un-catalysed reaction (Scheme 4 and Table 2).

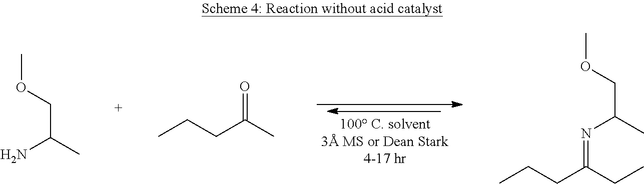

TABLE 2
Results from uncatalyzed imine formation experiments

| | Reaction | Conversion | Isolated |
|---|---|---|---|
| Drying method | Time (hr) | (%, GC area) | yield (%) |
| 3 Å molecular sieves in reactor | 4 | 63 | 34 |
| 3 Å molecular sieves in reactor | 18 | 68 | 36 |
| Dean Stark trap (containing 3 Å | 4 | 58 | 36 |
| molecular sieves) | | | |

End-of-reaction conversions were somewhat lower without added catalyst, but the reduced imine hydrolysis during workup resulted in improved isolated yield of ca. 35%.

### Trifluoroacetic Acid:

Previous literature reports by Meyers et al. had shown that excellent conversions could be achieved with structurally similar substrates when TFA was used as catalyst (Scheme 5). Work-up in this case involved addition of solid potassium carbonate to neutralise the acid followed by filtration. This approach avoids the challenges associated with aqueous workup and so was evaluated experimentally (Scheme 6).

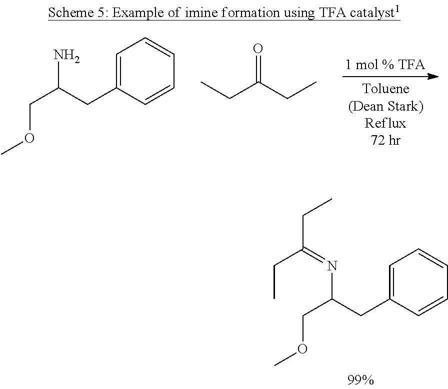

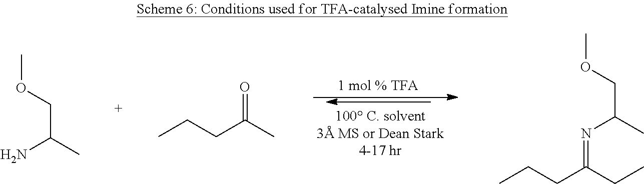

Both molecular sieve and Dean-Stark drying strategies were evaluated (Table 3). Dean-Stark conditions using TFA gave good results, with 85% conversion achieved in 4 hours. The work-up was also straightforward, with 45% isolated yield being reproducibly achieved. Isolated Imine quality had 85% strength with 3% unreacted ketone and 12% residual toluene.

TABLE 3
Results from TFA-catalysed imine formation experiments

| | Reaction | Conversion | Isolated | |
|---|---|---|---|---|
| Drying method | Time (hr) | (%, GC area) | yield (%) | Comment |
| 3 Å molecular sieves | 4 | 62 | 35 | |
| in reactor | | | | |
| Dean-Stark trap | 4 | 84 | 46 | Conversion dropped to ca. |
| (containing 3 Å | | | | 55% on standing overnight |
| molecular sieves) | | | | before work-up |
| Dean-Stark trap | 4 | 85 | 46 | Reaction worked-up on |
| (containing 3 Å | | | | same day as experiment |
| molecular sieves) | | | | |

Stability of the Imine product (stored over molecular sieves) was also assessed by quantitative ¹H NMR. TFA under Dean-Stark conditions is a preferred procedure for making the imine. This process provided a reliable method to generate reasonable yield of acceptable quality product.

Typical results obtained under Dean Stark conditions were ˜45% yield Imine with ˜90% purity. NMR & GC analysis indicated the bulk of the remaining mixture was toluene (˜7%) and starting 3-hexanone (˜3%).

### Step 2: Chloroacrolein Synthesis

2-chloroacrolein syntheses have been reported in the literature. One example, oxidation of 2-chloropropenol, is expedient method shown in Scheme 7.

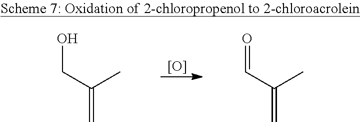

A Swern oxidation method for this transformation has also been reported in the literature. Dess-Martin oxidation is also as an alternative but carries safety concerns, e.g., low onset exotherm and explosive hydrolysis by-product. The Swern oxidation approach is shown in Scheme 8.

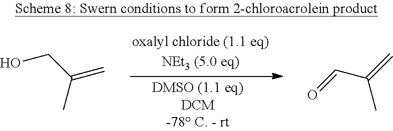

The best yield reported in the literature for this process is only 40%, suggesting the process may be challenging. The compound is relatively volatile so product loss during solvent removal is a potential problem. The product is also reported to have relatively poor stability. Two stabilisation strategies had been used to address this: addition of hydroquinone (to suppress radical reactions), and storing in a freezer at −80° C.

Running the standard Swern process gave >80% conversion to the desired product (confirmed by 1H, ¹³C NMR and GC-MS data), the main contaminants being residual DMS, DMSO, triethylamine and DCM. Product stability was assessed under a range of conditions. Storing at −20° C. resulted in a 30% drop in strength after one week. Product loss after 1 week at −80° C. was significantly worse (50%) but may have been an artefact of warming the material to room temperature before sampling (to avoid moisture ingress).

¹H NMR) suggested that two new aldehydic species had been formed (doublets either side of the singlet for the desired product). This evidence was also supported by the presence of two additional peaks in the aldehyde region of ¹³C NMR spectrum.

Tolerable stability of 2-chloroacrolein had been demonstrated (5% strength loss per day if stored at −20° C.), and 35% isolated yield of 73% str 2-chloroacrolein (quant ¹H NMR) achieved. Careful storage and prompt consumption of the aldehyde is needed.

Subsequently prepared chloroacrolein was stabilised by addition of hydroquinone in addition to storage in the freezer.

### Step 3: NAA Synthesis

The presented technology provide for a novel transformation to produce NAA as shown in Scheme 9.

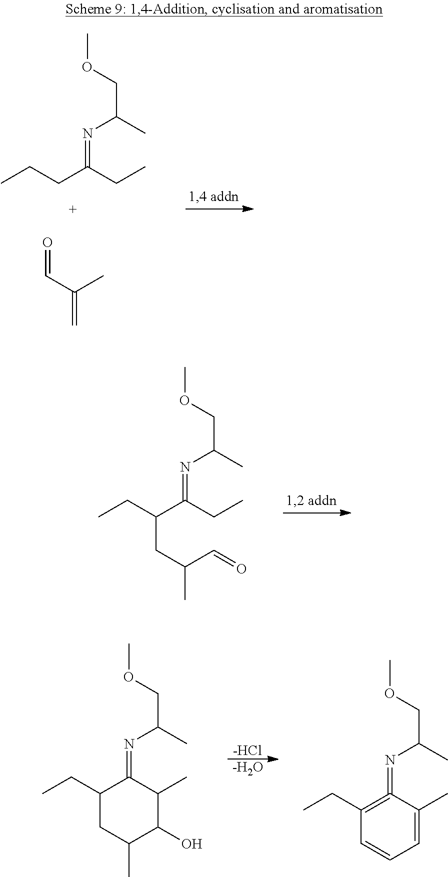

Both base- and acid-catalysed conditions were explored to effect this transformation. Base-catalysed conditions in chloroform solvent were initially investigated (Scheme 10 and Table 4).

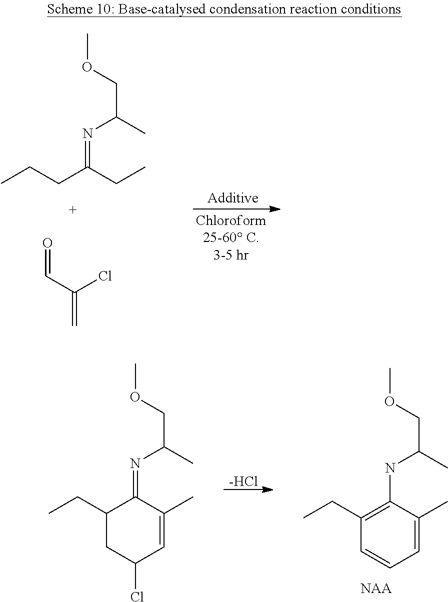

Reaction in the presence of proline, DBU and triethylamine however gave a trace of NAA (by GC).

TABLE 4
Base-catalysed condensation reactions

| | | | % Yield of | |
|---|---|---|---|---|
| Solvent | T (° C.) | Catalyst (eq) | NAA | Notes |
| Chloroform | 25 | Proline (0.1) | — | 1,4-addition product only by |
| | | | | MS. Column unsuccessful |
| Chloroform | 40 | DBU (0.5) proline (0.2) | Trace by GC | NAA + C₄H₈ observed by MS |
| Toluene | 40 | Triethylamine (1) | Trace by GC | NAA + C₄H₈ observed by MS |
| Toluene | 60 | DBU (1.0 eq), proline | — | |
| | | (50 mol %) | | |
| Toluene | 60 | NaOAc (1) | Trace by GC | Allyl chloride intermediate |
| | | | | detected by MS |

Results were also obtained with acid catalysis (Scheme 11 and Table 5).

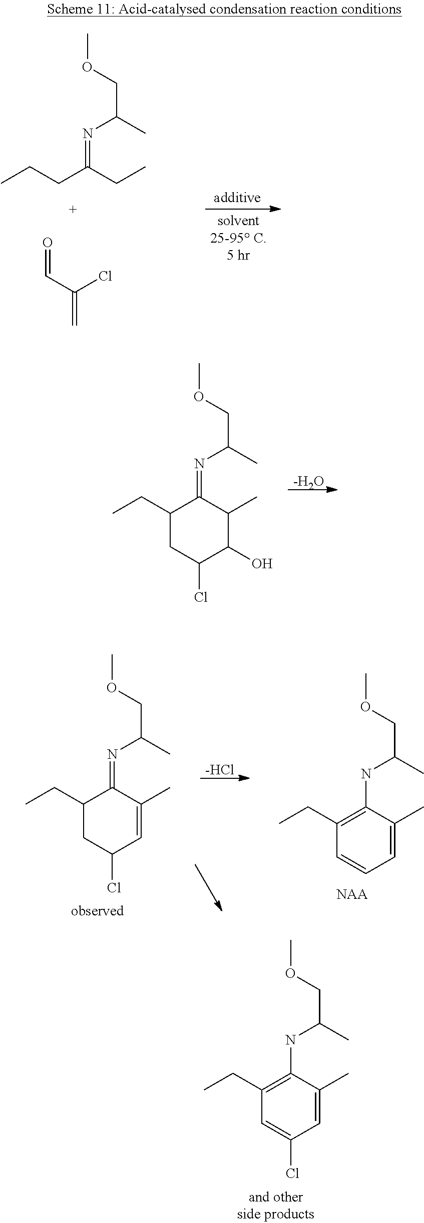

A range of conditions were investigated for this transformation; including different catalysts, solvents, temperature regimes, stoichiometries, and order of addition.

TABLE 5
Acid catalysed condensation reactions

| | | | | |
|---|---|---|---|---|
| | | | % Yield NAA | |
| Solvent | T (° C.) | Additive (eq) | (Quant-GC) | Notes |

Acetic acid catalyst in toluene solvent:

| | | | | |
|---|---|---|---|---|
| Toluene | 60 | AcOH (1) | 0.5 | NAA formation |
| | | | | detected by LCMS, |
| | | | | GCMS, GC |
| Toluene | 60 | AcOH (1) added last | 1 | |
| | | after 30 min | | |
| Toluene | 90 | AcOH (1) | 3 | |
| Toluene | 90 | AcOH (5) | 2 | |
| Toluene | 60 then 90 | AcOH (1) | 4 | |
| Toluene | 95 | AcOH (1) | 4 | Addition of |
| | | | | chloroacrolein at 95° C. |
| Toluene | 60 then 95 | AcOH (5) | 2 | |
| Toluene | 60 then 95 | AcOH (0.2) | 6 | |
| Toluene | 60 then 95 | AcOH (1) | 5 | Addition of |
| | | | | chloroacrolein at 60° C. |
| Toluene | 60 then 95 | AcOH (1) | 5 | Chloroacrolein added |
| | | | | dropwise |
| Toluene | 60 then 95 | AcOH (1) added last | 11 | Fresh imine used. Yield |
| | | after 30 min | | determined by Q-NMR |
| Toluene | RT then 95 | AcOH (1), molecular | 12 | 2 equivalents of |
| | | sieves | | chloroacrolein added |

Alternative catalyst systems in toluene solvent:

| | | | | |
|---|---|---|---|---|
| Toluene | 60 then 95 | MSA (1) | 0 | |
| Toluene | 60 then 95 | TiOiPr₄ (1) | 1 | Lewis acid catalyst |
| Toluene | RT then 60 | Hexafluoroisopropanol | 1 | |
| | | (1) | | |
| Toluene | 60 then 95 | TFA (1) | 2 | Yield determined by |
| | | | | Q-NMR |
| Toluene | 60 then 95 | None | 3 | |
| Toluene | RT then 95 | AcOH (1) NaOAc (1) | 3 | Sodium acetate may be |
| | | | | insoluble |
| Toluene | RT then 95 | TBA OAc (1) | 6 | |
| Toluene | RT then 95 | Monosodium | 3 | Sodium phosphate may |
| | | phosphate (1) | | be insoluble |
| Toluene | RT then 95 | Benzoic acid (1) | 4 | |

Alternative solvent systems:

| | | | |
|---|---|---|---|
| AcOH | RT then 95 | None | 0 |
| MeCN | 60 | AcOH (1) | 1 |
| MeCN | 60 | AcOH (1) then NEt₃ (2) | 2 |
| | | after 2 hr | |
| DMF | RT then 95 | AcOH (1) | 4 |
| | then 130 | | |
| NMP | RT then 95 | AcOH (1), Molecular | 4 |
| | | sieves | |
| Propylene | RT then 95 | AcOH (1), Molecular | 9 |
| carbonate | | sieves | |

A small yield of NAA (3%) was seen in the absence of any catalyst, and improved results were obtained in the presence of acetic acid. Yield of the acetic acid/toluene system seemed to be relatively insensitive to the process changes employed.

Varying acetic acid stoichiometry (0.2 to 5 equivalents) had some effect, with various results being observed under catalytic conditions. Yield of NAA (11-12%) was obtained with 1 equivalent of acetic acid.

Use of fresh imine and chloroacrolein is theorised to be important for good performance due to their instability.

Yield determination was conducted by quantitative ¹H NMR relative to a trimethoxybenzene internal standard, or via calibrated GC versus trimethoxybenzene internal standard.

The desired transformation generates one equivalent of water (from dehydration of an alcohol intermediate), so presence of molecular sieves should in theory therefore be beneficial.

Use of other acid catalysts with a range of different pKa's gave poorer results than acetic acid. The complexity of the overall transformation to NAA means that individual steps may have different reagent requirements-such as acid-catalysed dehydration and base-catalysed dehydrochlorination. Some mixed acid-base systems were therefore also studied (e.g., AcOH/NaOAc). Significant yield improvement was not observed, though poor solubility of the ionic materials was initially thought to be a factor a factor. However, use of fully soluble AcOH/Bu₄N⁺ ⁻OAc also failed to give any performance increase.

Several other significant products are visible by GC. These have been subjected to LC-MS and GC-MS by Helene Fain to identify the individual components. Initial results suggest that some of these are intermediates on the pathway to NAA, such as the allylic chloride in Scheme 9. However, some of the species look to be formed via competing pathways, such as Hantzsch pyridine synthesis.

### Intermediate to NAA: Examples of Side Products Formed:

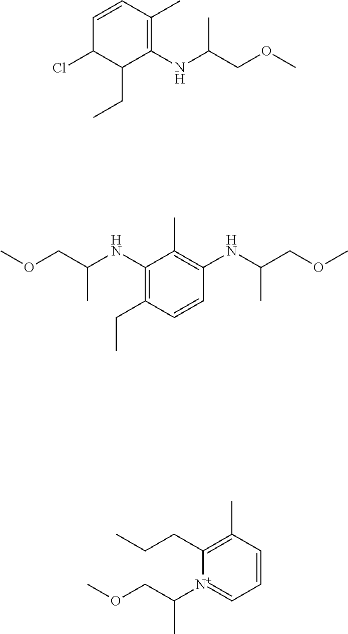

### Other Reaction Products Suggested by MS Analysis

### Chiral Synthesis

In another embodiment of the technology stereospecific NAA, here (S)-NAA is produced by via a route starting with a chiral amine as shown in Scheme 12.

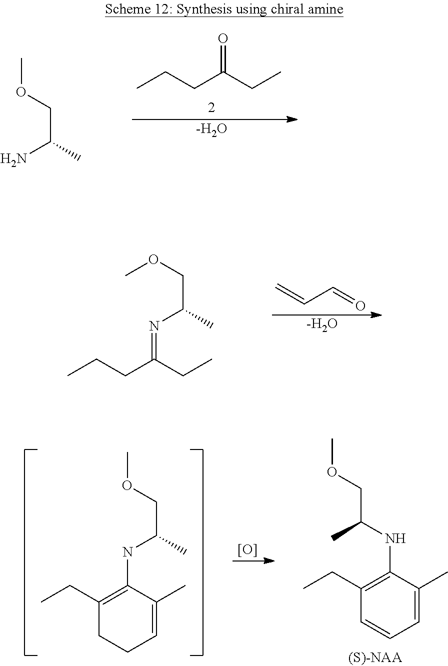

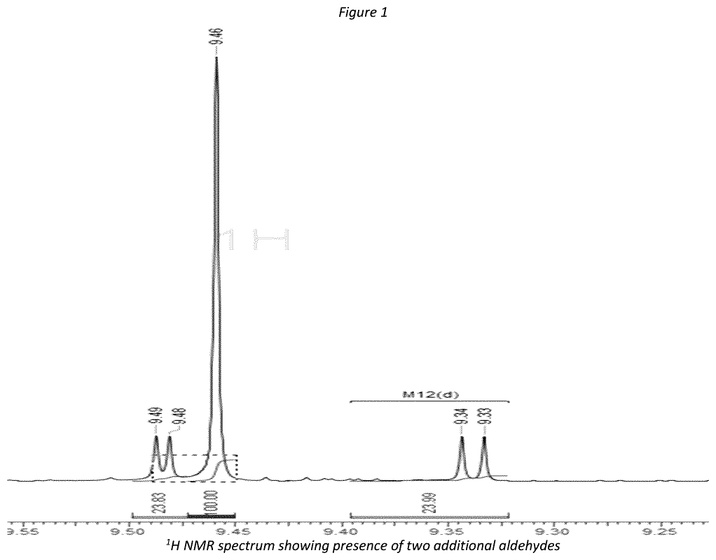

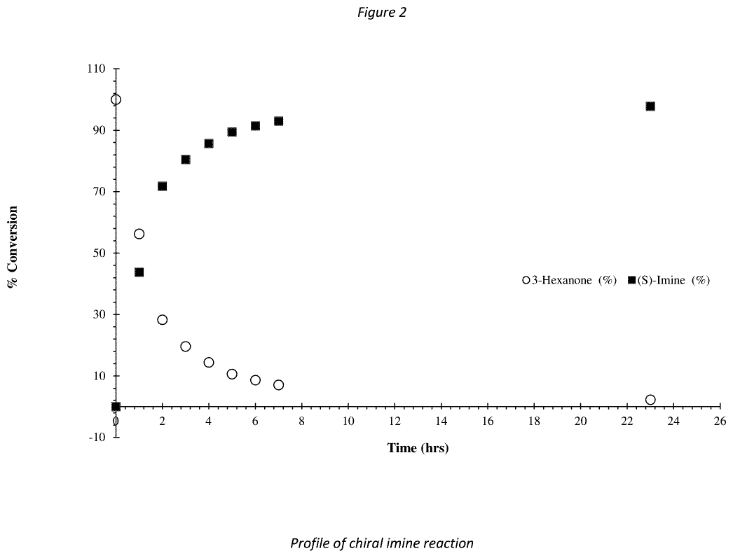

When chiral (S)-1-methoxy-2-propylamine (99% e.e.) was employed using the conditions described above, a slightly better yield of imine was achieved (52%). Reaction monitoring indicated ˜92% conversion was obtained in 7 hours, with final conversion of 98% after 23 hours (FIG. 2).

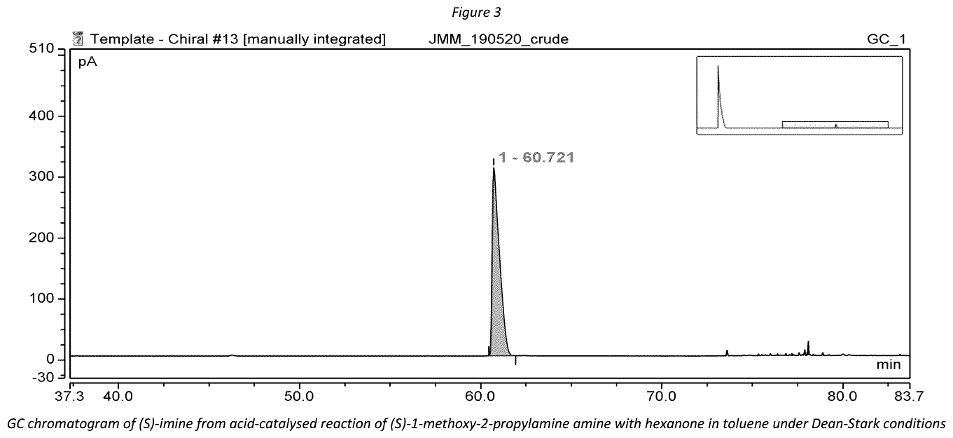

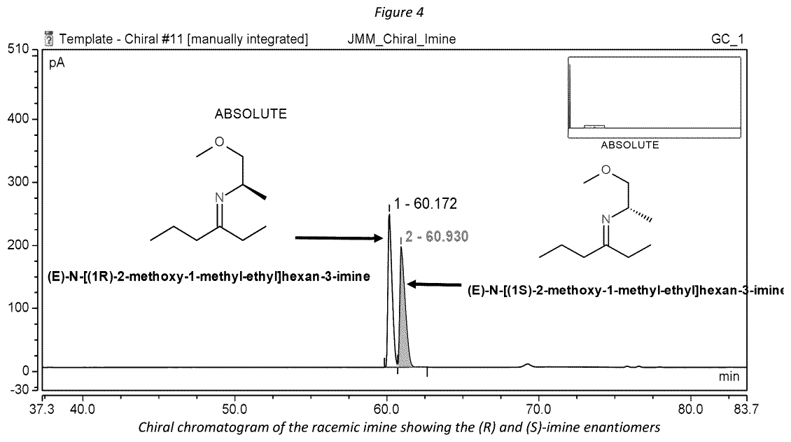

Chiral analysis of the final imine product showed excellent enantio-retention in the process (FIGS. 3 and 4).

Analysis of racemic imine gave two peaks, one at 60.93 for the (S)-imine and one at 60.17 for t®(R)-imine (FIG. 3). Analysis of the chiral reaction showed only one enantiomer, corresponding to (S)-imine.

### (S)-NAA Synthesis

(S)-imine was reacted with 2-chloroacrolein under the preferred conditions described earlier, to determine the enantio-integrity of the (S)-NAA forming step (Scheme 13).

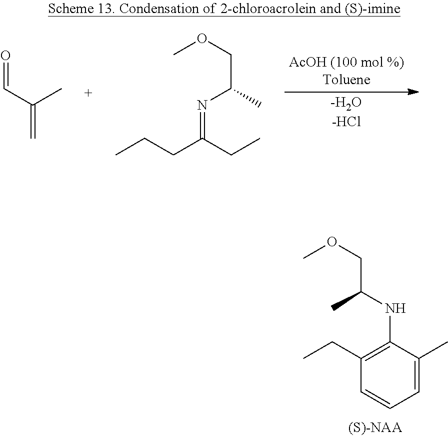

### GC Analysis:

A chiral GC method was developed for the analysis of the final product (S)-NAA, to integrate with analysis for the (S)-imine. Initial reactions used 1,3,5-trimethoxybenzene as the internal standard to assist yield quantification; but this co-eluted with the unwanted enantiomer (R)-NAA.

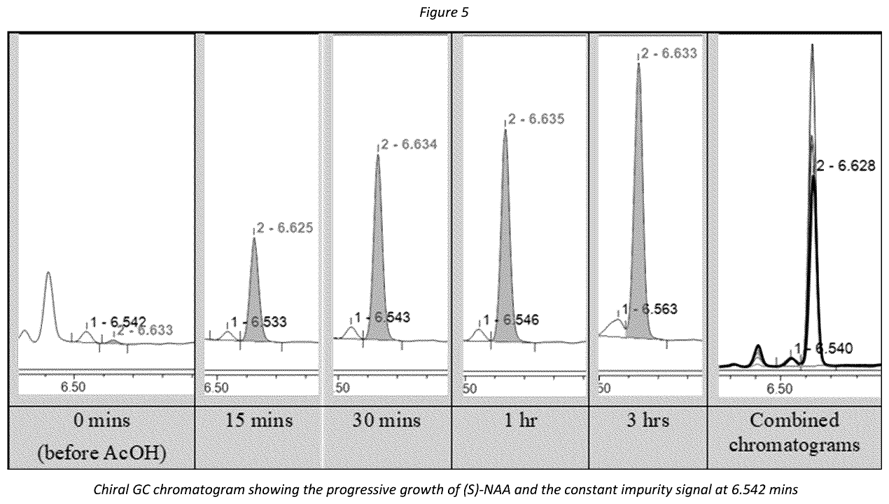

Omission of the internal standard gave better results but showed that an impurity present throughout the reaction (6.5 mins) also co-ran with R-amine (FIG. 5).

The amount of impurity present was relatively low (˜5%), but its presence meant that enantiomeric ratios measured by this method would be underestimated by that amount.

### LC-MS Analysis:

An alternative LC-MS method was therefore used to determine enantiopurity of the product. A sample of plant (S)-NAA was analysed using the following method:
Chromatograoph: Agilent
Detector: DAD
Column: Chiracel-OD (5 μm), 250 mm length, 4.6 mm i.d (Supplier: Daicel)
Column Temperature: room temperature
Flow rate: 1.0 ml/minute
Duration of chromatography: 20 minutes
Eluent: 2.0 mL isopropanol in 1000 ml hexane

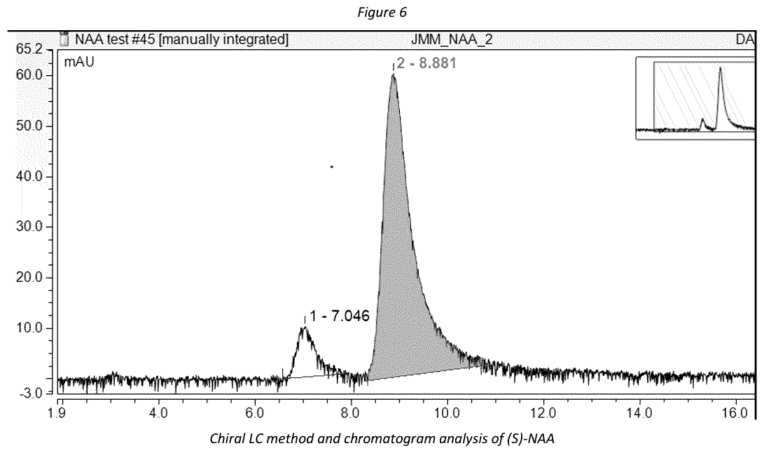

### [(R)-NAA-6.9 Mins and (S)-NAA-8.6 Mins] (FIG. 6).

Condensation of the (S)-imine and 2-choloroacrolein was monitored by this LC method. This analysis showed that, whilst some other low-level impurities were present, (S)-enantiomer was essentially the sole NAA product formed, with final (S)-NAA e.r of at least 99.3%. It also confirmed enantio-retention was excellent across the entire route with no evidence of racemisation of the chiral centre.

This three-step route has considerable scope for further optimisation now that its viability has been established.

### Acrolein Routes

Success of the chemistry with 2-chloroacrolein encouraged the exploration of a related route idea which used acrolein as substrate (Scheme 14).

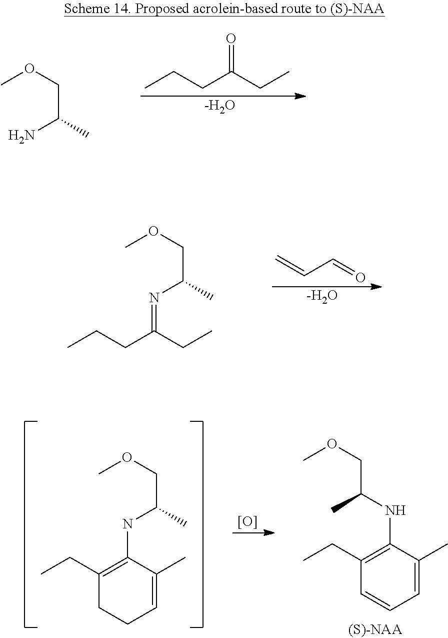

Acrolein is a cheap bulk chemical and so a potentially attractive raw material. Given the relative oxidation states of the substrates, acrolein would be expected to form the corresponding dihydro-aniline (in contrast to chloroacrolein which forms (S)-NAA directly).

The key ring-forming step envisaged imine/enamine tautomerism, 1,4-addition to the acrolein, cyclisation via 1,2-addition, followed by dehydration to afford the desired dihydro-aniline intermediate. The aromaticity driving force should then facilitate oxidation of the dihydro-aniline to NAA.

### Acrolein Dimethyl Acetal.

Though available at bulk scale, transportation hazards limit acrolein availability at lab scale. The reaction was therefore initially tested on the related more available material, acrolein dimethyl acetal (Scheme 15).

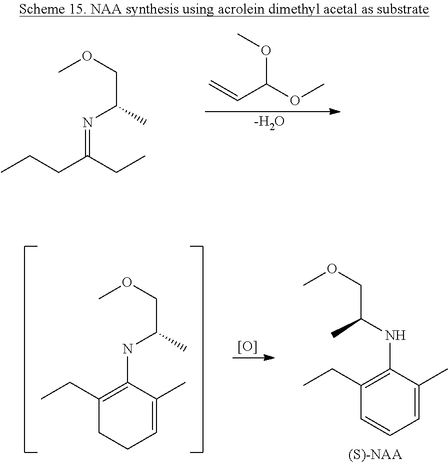

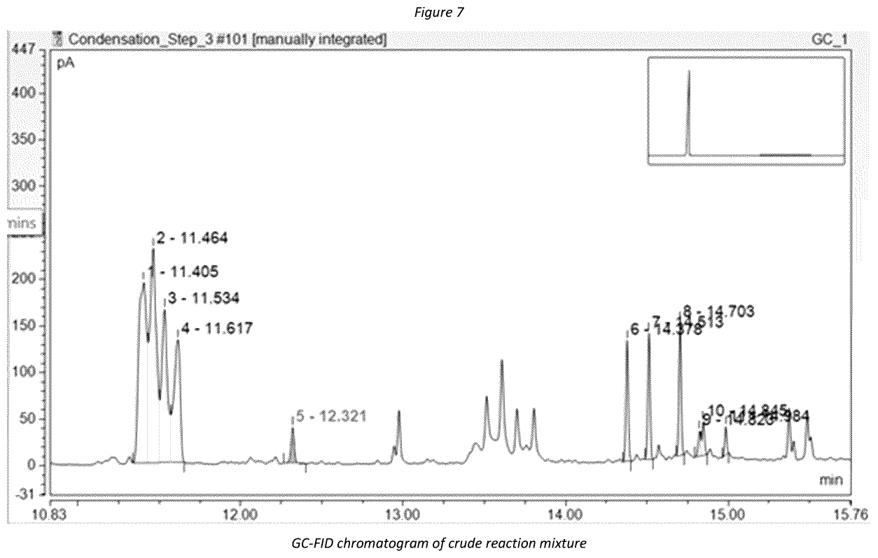

The transformation was initially attempted without any oxidant present, and with racemic imine to preserve stocks of the more precious chiral imine. The process involved heating imine and acrolein dimethyl acetal with trifluroacetic acid in toluene. End-of-reaction analysis indicated a trace amount of NAA had been formed along with a number of other major products, in particular a cluster of peaks with GC retention times between 11.464-11.617 minutes (FIG. 7).

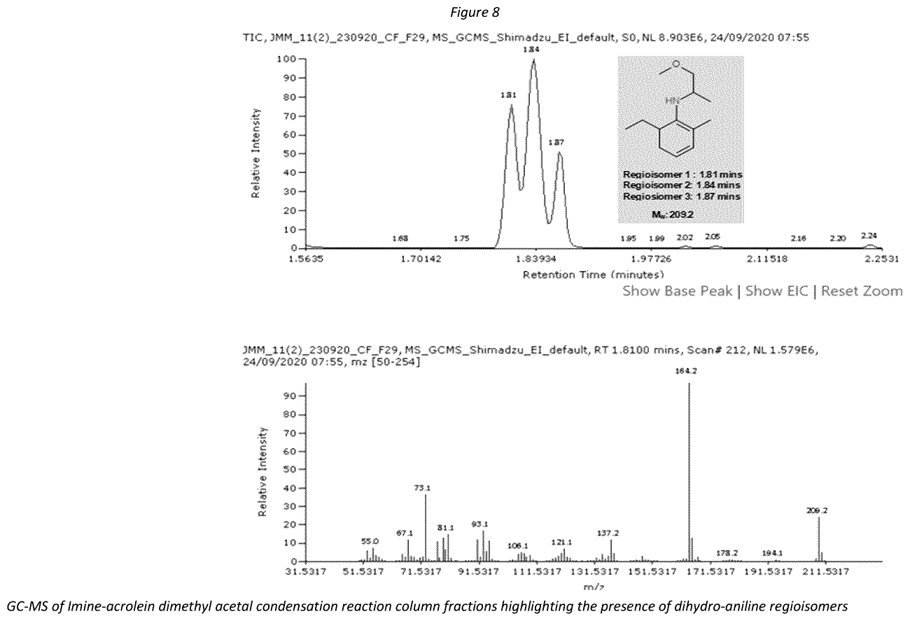

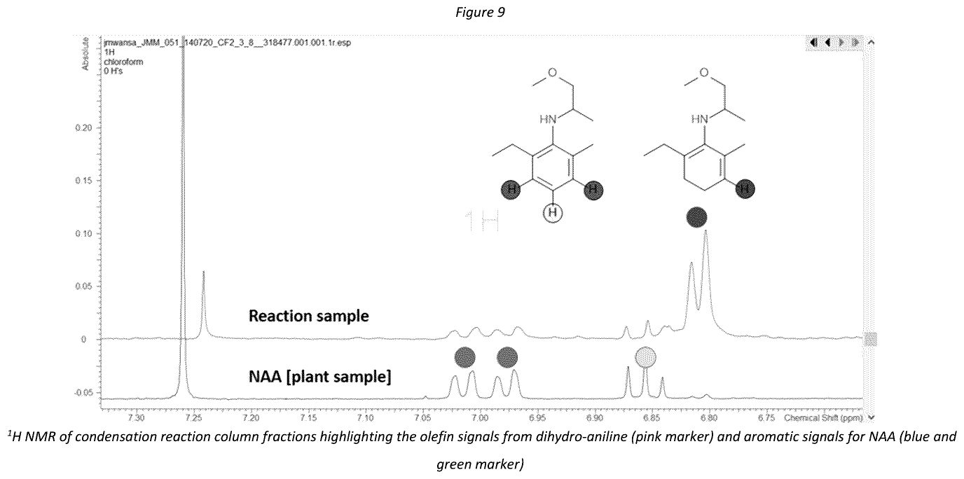

Combiflash chromatography and GC-MS analysis of the fractions showed this cluster of signals to be various isomeric forms of the required dihydro-aniline (FIG. 8). NMR also showed olefin signals consistent with these dihydro-aniline species (FIG. 9). Quantitative NMR analysis of the crude product from a repeat experiment estimated the yield of the dihydro-aniline intermediate species to be ˜20%.

A repeat of this reaction but in the presence of Cu(OAc)₂ oxidant readily converted the dihydro species to NAA in-situ. Good and clean conversion directly to NAA was achieved within 60 minutes, with no intermediate dihydro-species being observed.

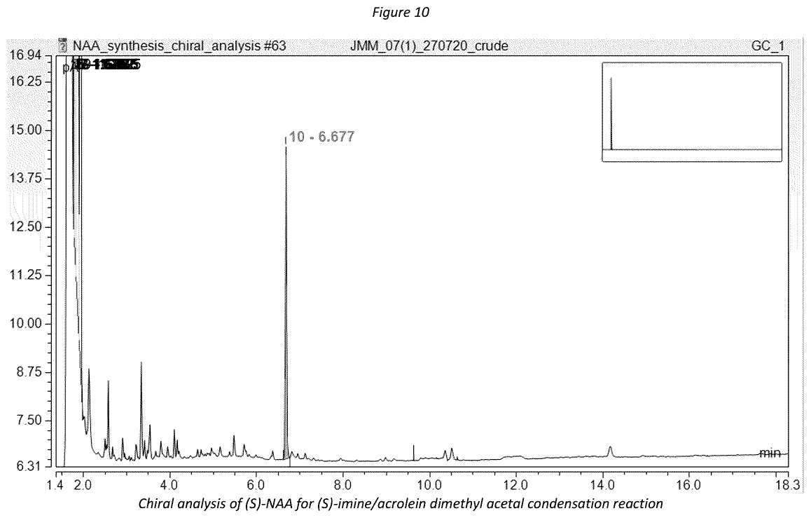

Repeat of the experiment with chiral (S)-imine gave similar performance. Chiral analysis of the product from this step showed e.r for (S)-NAA of 99.8%, confirming that the enantio-integrity is maintained with acrolein dimethyl acetal as substrate (FIG. 10). This also showed that excellent chiral retention had been achieved across the entire route to (S)-NAA.

### Acrolein

The above TFA-catalysed condensation reaction of chiral (S)-imine was repeated with acrolein as the substrate (Scheme 16).

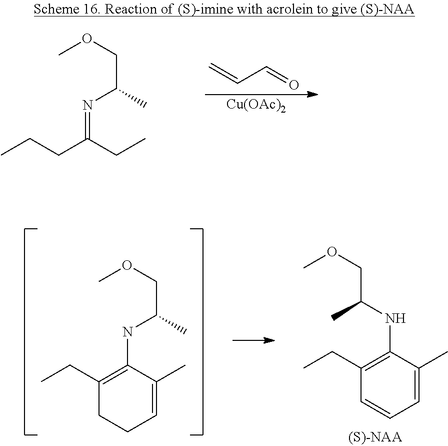

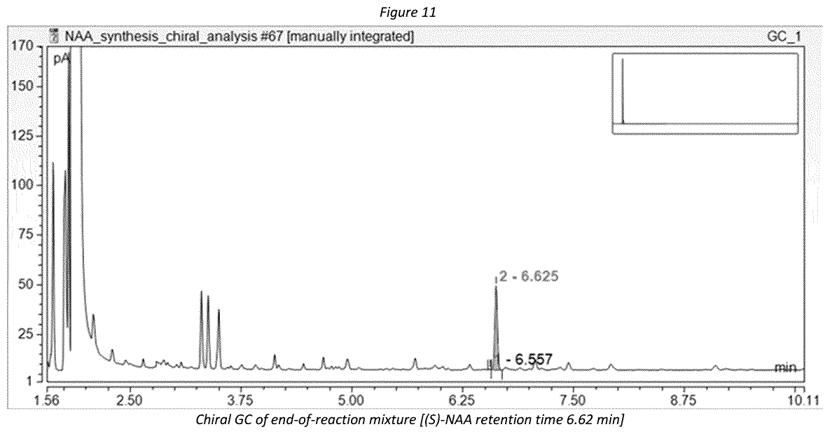

Conversion of 60% was achieved in 3 hrs to give the desired product (S)-NAA, confirmed by GC-FID and GC-MS. Chiral analysis by GC-FID showed product e.r. to be >99% (FIG. 11). Replication of this result showed good reproducibility, with (S)-NAA e.r. of 99.9.

The invention is defined by the claims.

1. A method of producing a compound formula (I) 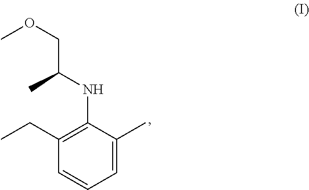
the method comprising: reacting a compound of formula (II) 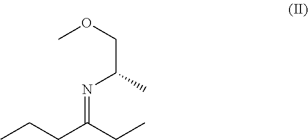
with a compound of formula (III) 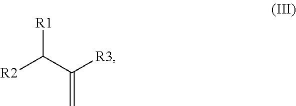
wherein,
R3 is hydrogen or halogen;
R1 and R2 are each independently C₁-C₆alkoxy, or R1 and R2 together are ═O or —OCH₂CH₂O— or —OCH₂CH₂CH₂O—; and
optionally whereby the reaction is carried out in the presence of an oxidizer.

2. The method of claim 1, wherein
R3 is Cl or Br; and
R1 and R2 are each independently dimethoxy, diethoxy, ethylene glycol, or R1 and R2 together are ═O.

3. The method of claim 1, wherein the compound of formula (III) is

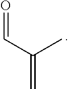

4. The method of claim 1, wherein the compound of formula (III) is acrolein or acrolein dimethyl acetal.

5. The method of claim 1, wherein an oxidizer is present.

6. The method of claim 5, wherein the oxidizer is copper acetate.

7. A method of producing s-metolachlor, the method comprising: reacting a compound formula (I) 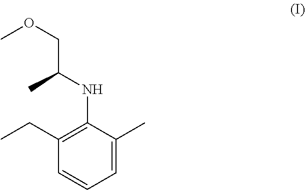
with chloroacetyl chloride, where the compound of formula (I) was produced by the method of any of claim 1.

8. A method of producing a compound formula (IV) 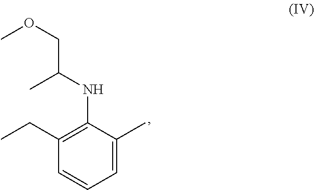
the method comprising: reacting a compound of formula (V) 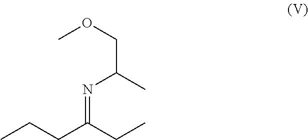
with a compound of formula (VI) 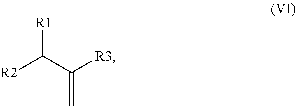
wherein,
R3 is hydrogen or halogen;
R1 and R2 are each independently C₁-C₆alkoxy, or R1 and R2 together are ═O or —OCH₂CH₂O— or —OCH₂CH₂CH₂O—; and
optionally whereby the reaction is carried out in the presence of an oxidizer.

9. A method of producing metolachlor, the method comprising:
reacting a compound of formula (IV) 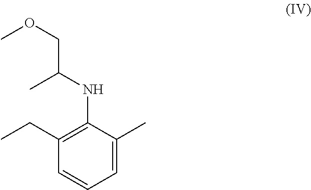
with chloroacetyl chloride, where the compound of formula (IV) was produced by the method of claim 8.
